(19) United States
(12) Patent Application Publication
(10) Pub. No.: US 2018/0214372 A1
(43) Pub. Date: Aug. 2, 2018

(54) EFFERVESCENT FORMULATIONS OF ORNITHINE ASPARTATE

(71) Applicant: Evonik Technochemie GmbH, Dossenheim (DE)

(72) Inventors: Shraddha Sanjeev JOSHI, Navi Mumbai (IN); Ashish Sharadchandra GUHA, Mumbai, Dombivali (E) (IN); Kedar GADILKAR, Ahmednagar (IN); Mandar MANOLIKAR, Banglaore, Karnataka (IN)

(73) Assignee: Evonik Technochemie GmbH, Dossenheim (DE)

(21) Appl. No.: 15/746,989

(22) PCT Filed: Jul. 19, 2016
(86) PCT No.: PCT/EP2016/067163

§ 371 Date: Jan. 23, 2018

(30) Foreign Application Priority Data
Jul. 24, 2015 (IN) 3814/CHE/2015
Sep. 24, 2015 (EP) 15186592.0

(51) Int. Cl. A61K 9/46; A61K 9/00; A61K 9/16; A61K 9/20; A61K 31/198; A61K 47/02; A61K 47/22

(52) U.S. Cl. CPC A61K 9/0007; A61K 9/009; A61K 9/1682; A61K 47/22; A61K 31/198; A61K 47/02; A61K 9/2095

(57) ABSTRACT

The present invention relates to the field of pharmaceutical and nutraceutical formulations, and, more specifically, to effervescent formulations of ornithine aspartate as well as processes for their manufacture. Formulations of ornithine aspartate according to the present invention comprise a gas generating component as well as an acid component releasing carbon dioxide upon contact with water. The invention provides a simple process for the manufacture of such formulations that are chemically pure and stable and exhibit high levels of effervescence. The process comprises the following steps: —granulation of an ornithine aspartate-mix comprising ornithine aspartate and a gas generating component, thus yielding granules G, —mixing the components of a final mix comprising granules G and an acid component, thus yielding an effervescent formulation of ornithine aspartate.

### FIELD OF THE INVENTION

The present invention relates to the field of pharmaceutical and nutraceutical formulations. More specifically, the present invention relates to effervescent formulations of ornithine aspartate as well as processes for their manufacture.

### BACKGROUND

In order to enable disintegration with sufficient intensity and generate enough effervescence to provide good taste and flavor, effervescent formulations must contain significant amounts of gas generating agents as well as acids. Effervescent formulations of ornithine aspartate containing such gas generating agents and acids, however, were found to be prone towards chemical degradation during manufacture and/or storage.

CN103860517A addresses this problem. The document discloses a method aimed at avoiding the formation of chemical impurities by employing melt granulation of sodium bicarbonate and polyethylene glycol (PEG 6000). The product obtained, thus, is mixed with granules of ornithine aspartate co-granulated with tartaric acid using a non-aqueous process beforehand. The process disclosed in CN103860517A, however, exhibits a number of disadvantages: Melt granulation is a relatively complex process necessitating use of specialized equipment. Further, the process does not allow manufacture of formulations with low levels of impurity. Additionally, the process cannot incorporate high contents of gas generating agents and acids, thus, merely yielding formulations with a less than desirable level of effervescence.

### DETAILED DESCRIPTION

The problem underlying the present invention, thus, resides in providing a simple process for the manufacture of chemically pure and stable formulations of ornithine aspartate with high levels of effervescence.

This problem is solved by the present invention providing a process for the manufacture of an effervescent formulation of ornithine aspartate comprising the following steps:
granulation of an ornithine aspartate-mix comprising ornithine aspartate and a gas generating component, thus yielding granules G,
mixing the components of a final mix comprising granules G and an acid component, thus yielding an effervescent formulation of ornithine aspartate.

According to the process of the present invention granulation of ornithine aspartate is performed in the presence of a gas generating component with no substantial amounts of acids present. Minor amounts of acids present during this step however, have no significant detrimental impact. A person of skill in the art, relying on impurity measurements and stability studies as described in the present specification, will be able to determine suitable maximal levels of acids that can be present during this step in addition to the gas generating component. In other embodiments granulation of the ornithine aspartate-mix comprising ornithine aspartate and a gas generating component, yielding granules G, is performed with a molar ratio R1=n(total amount of substance of gas generating agents forming the gas generating component)/n(total amount of substance of pharmaceutically acceptable acids forming the acid component) selected from the following: R1>10, R1>20, R1>30, R1>100. In another embodiment of the present invention no pharmaceutically acceptable acids forming the acid component are present during this step. In yet another embodiment of the present invention no pharmaceutically acceptable acids are present during this step.

Similarly, according to the process of the present invention mixing the components of the final mix comprising granules G and an acid component, thus yielding an effervescent formulation of ornithine aspartate is performed with no substantial amounts of gas generating agents added in addition to the gas generating agents added with granules G.

Minor amounts of gas generating agents added in addition to the gas generating agents added with granules G during this step however, have no significant detrimental impact. A person of skill in the art, relying on impurity measurements and stability studies as described in the present specification, will be able to determine suitable maximal levels of gas generating agents that can be added in addition to the gas generating agents added with granules G during this step. In other embodiments mixing the components of the final mix comprising granules G and an acid component, thus yielding an effervescent formulation of ornithine aspartate is performed with a molar ratio R2=n(total amount of substance of pharmaceutically acceptable acids forming the acid component)/n(total amount of substance of gas generating agents added in addition to the gas generating agents added with granules G during this step) selected from the following: R2>10, R2>20, R2>30, R2>100. In another embodiment of the present invention no gas generating agents forming the gas generating component are added in addition to the gas generating agents added with granules G during this step. In yet another embodiment of the present invention no gas generating agents are added in addition to the gas generating agents added with granules G during this step.

L-ornithine-L-aspartate is the salt of L-ornithine and L-aspartic acid. In healthy individuals fed with a proper diet, L-ornithine and L-aspartate are synthesized de novo in sufficient quantities, but in certain states of disease, as a result of tissue damage, organ insufficiency, excessive metabolic demand, growth, pregnancy, or deficiencies of urea cycle enzymes, it was found that supplementing these amino acids had beneficial effects. Both amino acids play key roles in ammonia detoxification and in proline and polyamine biosyntheses. Polyamines are considered critical for DNA synthesis and cell replication and have been shown to stimulate hepatic regeneration. Supplementation with ornithine in animal models demonstrated enhanced wound breaking strength and collagen deposition. It has been shown in vitro, in vivo and in perfused organs that urea synthesis from ammonia is limited by endogenous ornithine and that ornithine supplementation can promote urea formation to a significant degree. Low and high dose formulations of L-ornithine-L-aspartate are currently being marketed. Low dose formulations are used primarily as food supplements while high dose formulations (above 5 g) are used for example for lowering blood ammonia concentration and for eliminating symptoms of hepatic encephalopathy associated with liver cirrhosis. (Pol Merkur Lekarski. 2010 June; 28(168):490-5).

Effervescent formulations are intended to disintegrate fast, and rapidly and simultaneously release the active ingredients contained therein into an aqueous fluid. They comprise a mixture of ingredients (gas generating component and acid component) which release carbon dioxide upon contact with water. Effervescent formulations according to the present invention may further comprise additional pharmaceutically acceptable ingredients, such as excipients and coadjuvants selected from viscosity modifiers, fillers, disintegrants, lubricants, diluents, binders, glidants, antifoaming agents, wetting agents, colors, sweeteners and flavourings.

Disintegrants support rapid disintegration of tablets in aqueous fluids. Disintegrants increase the surface area of tablets in water rapidly disintegrating the tablet into small particles. Polymers which have a high degree of disintegration power include, inter alia, cross linked sodium carboxymethylcellulose, cross-linked hydroxypropylcellulose, high molecular weight hydroxypropylmethylcellulose, carboxymethylamide, potassium methacrylatedivinylbenzene copolymer, polymethylmethacrylate, cross-linked polyvinylpyrrolidone, high-molecular weight polyvinyl alcohols, microcrystalline cellulose, and the like. Particular examples of disintegrants are sodium starch glycolate, polymeric derivatives of acrylic acid, crosprovidone, and microcrystalline cellulose.

Fillers or diluents facilitate compression of powder and have an influence on the hardness of a tablet after compression. Furthermore they adjust the volume for potency. Such compounds comprise polyols, celluloses, starch and its derivatives, Lactose, isomalt, maltodextrin.

Lubricants are excipients which reduce inter-particle friction inside a tablet and reduce the reaction forces appearing on the die wall during compression or compaction. Lubricants are for example talcum, stearyl fumarate, polyethylene glycol, salts of benzoic acid, such as the sodium or lithium salt, L-leucine and magnesium stearate.

Flavouring agents (flavors) contribute to the taste for example the taste of a natural fruit, such as orange, lemon, apple, strawberry, vanilla, berries or of a herb, for example peppermint, or of broiled or fried meat, such as extracts from liver or yeast.

Sweetening agents are for example, saccharin, aspartame, cyclamate, sorbitol, sugar, polyols and mixtures thereof.

Colouring agents serve to give a pleasant appearance. Such agents are selected from any of the pharmaceutically or nutraceutically acceptable colors approved by regulatory agencies for example tartrazin (E102), crinoline yellow (E104), yellow orange (E110) and natural colors like anthocyanins.

Examples of binders are povidone, hydroxy propyl cellulose, carbomers, acrylic polymers, gums, PVA.

Examples of glidants are colloidal anhydrous silica, talc, L-leucine, stearates.

An example of an antifoaming agent is simitone.

An example of a wetting agent is polysorbate.

Effervescent formulations according to the present invention in the form of powders or granulates can be manufactured into numerous dosage forms including for example monolithic forms, such as tablets or pellets, as well as filled sachets. Tablet formulations comprising the effervescent formulations of the present invention may for example be formed by known compression pelleting techniques. In some cases dry densification processes may be used, e.g. briquetting, compression molding, and roller compaction.

In order to be administered the effervescent formulations of the present invention or dosage forms comprising the effervescent formulations of the present invention typically are dispersed in water or other aqueous fluids at room temperature, and administered orally. The amount of fluid is typically an amount that can conveniently be swallowed. For animals the formulations or dosage forms may be added to the food, or disintegrated in water and this form added to the food or injected into the mouth by means of a pipette.

The ornithine aspartate-mix granulated in the course of the process of the present invention comprises ornithine aspartate and a gas generating component. The product of this granulation are granules G. In addition to ornithine aspartate and the gas generating component the ornithine aspartate mix may comprise one or more of the following viscosity modifiers, fillers, disintegrants, lubricants, binders, antifoaming agents, wetting agents, colors, sweeteners and flavors.

According to the present invention Ornithine aspartate the salt of ornithine and aspartic acid is added to the ornithine aspartate mix. In the context of the present disclosure ornithine aspartate refers to L-ornithine L-aspartate, which is the salt of L-ornithine and L-aspartic acid.

The gas generating component according to the present invention consists of one or more gas generating agents. In the presence of the acid component and when contacted with water these gas generating agents release carbon dioxide. Accordingly, gas generating agents constituting the gas generating component are compounds releasing carbon dioxide when contacted with water in the presence of the acid component.

In other embodiments of the present invention the gas generating agents constituting the gas generating component are selected from the following: One or more carbonate salts, one or more bicarbonate salts, mixtures of one or more carbonate salts, mixtures of one or more bicarbonate salts, mixtures of one or more carbonate salts with one or more bicarbonate salts.

In other embodiments of the present invention the gas generating agents constituting the gas generating component are selected from the following: sodium carbonate, sodium bicarbonate, potassium carbonate, potassium bicarbonate, magnesium carbonate, calcium carbonate.

In other embodiments of the present invention the gas generating component consists of sodium bicarbonate.

In other embodiments of the present invention the total amount of gas generating agents constituting the gas generating component added to the formulation is sufficient to yield effervescent formulations of ornithine aspartate comprising 20 wt % to 40 wt % of gas generating component.

According to the present invention an ornithine aspartate-mix comprising ornithine aspartate and a gas generating component are granulated, thus yielding granules G,

Granulation of the ornithine aspartate-mix can be performed by any of several methods known in the art such as aqueous granulation, non-aqueous solvent based granulation, dry granulation, compaction, slugging, melt granulation, agglomeration by heat application and combinations thereof. Such methods for granulation are well established in the field and thus well known to a person of skill in the art. They have also been described in detail in the literature (cf. e.g. Aulton's Pharmaceutics: The Design and Manufacture of Medicines, Chapter: Pharmaceutical Technology of Granule production by Michael E. Aulton, Kevin M. G. Taylor, Part 5, pages 472-485; Handbook of Pharmaceutical, Granulation Technology, Executive Editor James Swarbrick, PharmaceuTech Inc. Pinehurst, N.C. 2005 by Taylor & Francis Group, LLC. Chapter, Theory Of granulation: An Engineering prospective pages 7-60).

In other embodiments of the present invention granulation conditions employed for granulation of the ornithine aspartate-mix are selected from the following: wet granulation, dry granulation, melt granulation.

In view of the fact that melt granulation is a relatively complex process necessitating use of specialized equipment, while the process of the present invention does not require melt granulation conditions to be used, in a specific embodiment of the present invention no melt granulation conditions are employed in the process.

According to the present invention the components of a final mix comprising granules G and an acid component, are mixed thus yielding an effervescent formulation of ornithine aspartate.

In addition to granules G and an acid component the final mix according to the present invention may comprise one or more of the following: Viscosity modifiers, fillers, disintegrants, lubricants, diluents, binders, glidants, antifoaming agents, wetting agents, colors, sweeteners and flavors.

Mixing of the final mix can be performed by any of several methods known in the art such as blending, high shear mixing, geometric mixing, tumbling, co-milling etc. Such methods for mixing are well established in the field and thus well known to a person of skill in the art. They have also been described in detail in the literature (cf. e.g. Powder Technology, Handbook, Marcel Dekker, New York, 1997, Pages 43-56; Pharmaceutical Blending and Mixing, 1st edition, edited by P. J. Cullen, Rodolfo Roma Ãach, Nicolas Abatzaglou, Chris D. Rielly. Willey Publication. Chapter 6, Continuous Powder Mixing. Pg. 102-484).

In other embodiments of the present invention mixing conditions employed for mixing of the final mix are selected from the following: Blending, high shear mixing, geometric mixing.

The acid component according to the present invention consists of one or more organic or inorganic pharmaceutically acceptable acids. In the presence of the acid component and when contacted with water the gas generating agents release carbon dioxide. Accordingly, pharmaceutically acceptable acids constituting the acid component are compounds inducing release of carbon dioxide when contacted with water in the presence of the gas generating component.

In other embodiments of the present invention pharmaceutically acceptable acids constituting the acid component are selected from the following acids as well as partial salts of the following acids with alkaline or alkaline earth metals in the case of polybasic acids: citric acid, tartaric acid, malic acid, adipic acid, succinic acid, fumaric acid, ascorbic acid, maleic acid, mixtures thereof.

In other embodiments of the present invention the acid component consists of citric acid.

According to the present invention pharmaceutically acceptable acids constituting the acid component are added in overall molar excess to the gas generating components constituting the gas generating component. Typically, pharmaceutically acceptable acids constituting the acid component and gas generating agents constituting the gas generating component are added to the formulation in an amount yielding effervescent formulations with a molar ratio of acid component to gas generating component in the range of 7:1 to 1.1:1.

An advantage of the process of the present invention resides in the fact that it can be performed at high levels of humidity, including humidity levels of up to 50% relative humidity.

The present invention further comprises effervescent formulations of ornithine aspartate obtainable by a process according to the invention.

In addition to ornithine aspartate, gas generating component and acid component effervescent formulations according to the present invention may further comprise one or more excipients selected from the following: Viscosity modifiers, fillers, disintegrants, lubricants, diluents, binders, glidants, antifoaming agents, wetting agents, colors, sweeteners, flavors.

In a specific embodiment of the present invention effervescent formulations according to the present invention contain one or more additional pharmaceutically acceptable ingredients selected from: fillers, lubricants, diluents, binders, glidants, colors, sweeteners, flavors.

Typically, effervescent formulations according to the present invention contain ornithine aspartate, gas generating component, acid component and additional pharmaceutically acceptable ingredients in the following percentages (for each specific formulation percentages must be selected to add up to 100%):

ornithine aspartate: 10%-60%

gas generating component: 10%-40%

acid component: 15%-50%

fillers/diluents: 0%-25%

lubricants/glidants: 0%-15%

binders: 0%-10%

sweeteners: 0%-10%

flavourings: 0%-2%

In a specific embodiment of the present invention effervescent formulations according to the present invention contain ornithine aspartate, gas generating component and acid component as well as the following pharmaceutically acceptable ingredients: Fillers, lubricants, diluents, binders, glidants, colors, sweeteners and flavors.

In another aspect the present invention provides a process for the manufacture of stable effervescent formulations of ornithine aspartate containing large amounts of gas generating agents. Formulations containing large amounts of gas generating agents are desirable for a number of applications because they are capable of imparting good palatability on drinks obtained therefrom by carbonation of the liquid and evolution of carbon dioxide in gaseous form. One reason for this effect is that dissolved carbon dioxide contributes to the taste directly by interacting with the sour taste buds. Furthermore, evolved carbon dioxide in gaseous form helps in improving the flavor and thus also contributes to the taste indirectly through olfactory sensing. Accordingly, the amount of carbon dioxide that can be produced by an effervescent formulation is an important factor and in the present context it is referred to as the gas generating capacity of an effervescent formulation. In order to impart beneficial taste properties to a drink obtained therefrom it is desirable to apply effervescent dosage forms exhibiting sufficient gas generating capacity to saturate the drink with carbon dioxide. In view of the fact that the typical volume of a drink obtained from an effervescent formulation is about 100 mL and, further, that 100 mL of water are capable of dissolving about 150 mg of carbon dioxide at room temperature (25° C.), a gas generating capacity of 150 mg carbon dioxide is usually sufficient to impart the corresponding beneficial taste properties.

Therefore, in one embodiment, the process of the present invention allows to obtain effervescent formulations of ornithine aspartate exhibiting a gas generating capacity of more than 150 mg carbon dioxide per gram of ornithine aspartate.

Accordingly, further, in one embodiments the present invention provides effervescent formulations of ornithine aspartate obtainable by a process of the present invention, exhibiting a gas generating capacity of more than 150 mg carbon dioxide per gram of ornithine aspartate.

As indicated above effervescent formulations according to the present invention in the form of powders or granulates can be manufactured into numerous dosage forms including for example monolithic forms, such as tablets or pellets, as well as filled sachets. Accordingly, in another embodiment the present invention comprises tablets, pellets or sachets comprising effervescent formulations of ornithine aspartate according to the present invention.

### DESCRIPTION OF FIGURES

FIG. 1 Flow chart of the manufacturing process

FIG. 2 Effervescence/CO₂ generation characteristics of sample-G_{I} in water (100 mL) at 25° C.

FIG. 3 Effervescence/CO₂ generation characteristics of samples H_{I}, J_{I}, N_{I} O_{C}, P_{I}, Q_{C} in water (100 mL) at 25° C.

FIG. 4 Effervescence/CO₂ generation characteristics of sample-V_{I} in water (100 mL) at 25° C.

### EXAMPLES

(1) Analytical Methodology

The following section describes the analytical methods used for analyzing samples A to V below.

Method for Impurity Analysis:

Chromatographic Conditions
Column: Waters Spherisorb Amino 5μ (250×4.6)mm
Mobile Phase: Buffer: Acetonitrile (27.5:72.5)
   Buffer: 0.05M KH2PO4
Wavelength: 210 nm
Column Temp: 25° C.
Injection volume: 100 μL
Flow rate: 1.0 mL/minute
Run time: 120 minutes
Sample Temp: Ambient
Solvents used were of HPLC grade.
Diluent: Mobile phase

Standard Preparation

L-Ornithine Lactam Impurity ((3S)-3-Aminopiperidin-2-one*Hcl) solution: 5 mg Impurity was weighed and transferred in 100 mL volumetric flask. 30 mL water was added and sonicated to dissolve. Diluted up to the volume with water. (Approx. Concentration—50 ppm)

Fumaric acid solution: 20 mg of Fumaric acid was weighed and transferred in 1000 mL volumetric flask. 300 mL water added and sonicated to dissolve. Diluted up to the volume with water. (Approx. Concentration—20 ppm)

Malic acid solution: 150 mg Malic acid was weighed and transferred in 100 mL volumetric flask. 30 mL water was added and sonicated to dissolve. Diluted up to the volume with water. (Approx. Concentration—1500 ppm)

L-Arginine solution: 50 mg L-Arginine was weighed and transferred in 100 mL volumetric flask. 30 mL water was added and sonicated to dissolve. Diluted up to the volume with water. (Approx. Concentration—500 ppm)

System Suitability Solution Preparation:

130 mg of L-Ornithine L-Aspartate API was weighed and transferred in 50 mL volumetric flask. 2 mL of each of the impurity stock solutions were transferred to the flask and diluted to volume with diluent. (Approx. concentrations: Lactam—2 ppm, Fumaric acid—0.8 ppm, Malic acid—60 ppm, L-Arginine—20 ppm, L-Ornithine L-Aspartate—2600 ppm)

Sample Preparation:

For sachets containing effervescent granules, entire contents of five sachets were emptied and weighed to obtain average weight of sachet. For effervescent tablets, five tablets were weighed to obtain average weight of a tablet and then crushed in to powder. An amount of effervescent formulation to be analyzed equivalent to 1000 mg of L-Ornithine L-Aspartate was weighed and transferred in 100 mL volumetric flask. 30 mL water was added and effervescence allowed to go away. Sonicated to dissolve and diluted up to the mark with water. This was stock solution containing about 10000 ppm of L-Ornithine L-Aspartate. Further, transferred 3 mL of stock solution in to a 10 mL volumetric flask and diluted up to the mark with mobile phase (Approx. L-Ornithine L-Aspartate concentration—3000 ppm). Filtered the solution through 0.45μ Nylon filter. First few mL of filtrate was discarded. This was sample solution used for injection in chromatographic system.

Placebo Preparation:

Placebo equivalent to 1000 mg of L-Ornithine L-Aspartate (Amount of Placebo=Average weight of sachet or tablet—L-Ornithine L-Aspartate content in each sachet or tablet (viz. 1000 mg)) was weighed and transferred in 100 mL volumetric flask. 30 mL water was added and effervescence allowed to go away. Sonicated to dissolve and diluted up to the mark with water. This was stock solution. Further, transferred 3 mL of stock solution in to a 10 mL volumetric flask and diluted up to the mark with mobile phase. Filtered the solution through 0.45μ Nylon filter. First few mL of filtrate was discarded. This was placebo solution used for injection in chromatographic system.

Procedure:

100 μl each of diluent, system suitability solution, Fumaric acid solution (0.8 ppm), and Placebo solution were injected into the chromatograph and the chromatograms were recorded. It was ensured that, system suitability parameters were fulfilled and there was no interference from the blank and placebo chromatograms at the retention time of the main peaks and impurity peaks.

The sample sequence for six batches typically followed was as given below. Same sequence was adopted for more than six batches.

| Sample name | No of injections | Type of testing |
|---|---|---|
| Diluent | 1 | Blank |
| System Suitability | 3 | System suitability and known |
| Solution | | impurity standard |
| Sample solution | 1 | Sample preparation of sample I |
| Sample solution | 1 | Sample preparation of sample II |
| Sample solution | 1 | Sample preparation of sample III |
| Sample solution | 1 | Sample preparation of sample IV |
| Sample solution | 1 | Sample preparation of sample V |
| Sample solution | 1 | Sample preparation of sample VI |
| Placebo | 1 | To check placebo interference |
| Diluent | 1 | Blank |
| System Suitability | 1 | System suitability and known |
| Solution | | impurity standard |

System Suitability:

The following system suitability criteria were measured form injection of System Suitability Solution: Theoretical plates for L-Aspartic acid peak are not less than 2000% RSD for area of Impurity peaks are not more than 3%.

Calculation:

% Individual Known Impurity in Sample

$$\% Individual Known Impurity in sample = \frac{A smp}{A std} \times \frac{W t std}{D stock} \times \frac{2}{50} \times \frac{100}{Wt smp} \times \frac{50}{5} \times \frac{Avg}{LC} \times 100$$

Where:
A smp: Area of Impurity obtained from measurement of sample solution
A std: Average area of Impurity obtained from a series of measurements of system suitability solutions
Wt std: Weight of Impurity standard in mg
Wt smp: Weight of sample in mg
D stock: Final dilution volume of impurity stock solution in mL
LC: L-Ornithine L-Aspartate content in each sachet or tablet in mg
Avg: Average weight of sachet or tablet in mg

Note: Calculation of known impurities was done using areas of individual impurity peaks as observed in system suitability solution.

% Unknown Impurity in Sample

$$\% Unk nown Impurity in sample = \frac{A smp}{A tot - A plc - A blk} \times 100$$

Where:

A smp: Area of Impurity in sample preparation

A tot: Total area in sample chromatogram

A plc: Area of placebo peaks in sample chromatogram

A blk: Area of blank peaks in sample chromatogram

% Individual Known Impurity+% Individual Unknown Impurity % Total Impurity in sample

% Total impurity×10000 Total Impurity in sample (in ppm)

Analytical Methodology for Determination of Effervescence Characteristics

Samples were placed in glass beaker containing 100 mL of demineralized water at a temperature of 25° C. The change in the total weight of contents was noted using a suitable digital balance over the period of 10 minutes. Total time required for visual disappearance of effervescence was also noted. The amount of CO₂ dissolved in water was arithmetically calculated by subtracting the amount of gas evolved in 10 minutes from the theoretical gas generating capacity.

(2) Formulation Details

List of Ingredients

Following is a list of the Excipients, manufacturers and specification, used in the examples below (USP=United States Pharmacopeia, BP=British Pharmacopeia):

TABLE 1
List of ingredients used

| S. No. | Name of excipient | Manufacturer/Supplier | Specification |
|---|---|---|---|
| 1 | L-ornithine L-Aspartate | Evonik Industries AG, | In House |
| | | Germany | |
| 2 | Povidone (PVPK 25) | Koje Polymer | USP |
| 3 | Color FD &C 6 Alum | Sensient | USP |
| | Lake | | |
| 4 | Sodium bicarbonate | Merck inc. | USP |
| 5 | Aspartame | Nutra Sweet. | USP |
| 6 | Flavor: Orange (501071) | Firmenich | USP |
| 7 | Citric acid Anhydrous | Sunil Pharma | BP |
| 8 | Polyethylene glycol | Sasol | USP |
| | (PEG 6000) | | |
| 9 | L-Leucine | Evonik Industries AG, | In House |
| | | Germany | |
| 10 | Isomalt (galen IQ 721) | Beneo-Palatinit gmbh | USP |

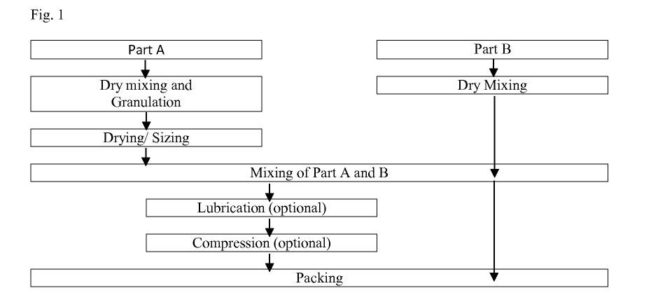

A Flow Chart of the Manufacturing Process is Displayed in FIG. 1.

Oral Formulations of L-Ornithine L-Aspartate

Suffixes denote type of example: _{C}=Comparative Example; _{I}=inventive Example

TABLE 2
Formula for examples A-F

| | Quantities | | | |
|---|---|---|---|---|
| | Example- | Example- | Example- | Example- |
| Ingredients | A_{C} | B_{C} | C_{C} | D_{C}, E_{C}, F_{I} |
| L-Ornithine L-Aspartate | 1000.0 | 1000.0 | 1000.0 | 1000.0 |
| Sodium bicarbonate | — | — | 950.00 | 950.00 |
| Citric Acid | — | 1525.0 | — | 1525.0 |
| Total weight | 1000.0 | 2525.0 | 1950.0 | 3475.0 |

TABLE 3
Formula for example G_{I}

| | | Weight per |
|---|---|---|
| | Ingredients | dose (mg) |
| Part-A | L-Ornithine L-Aspartate | 1000.0 |
| (L-Ornithine L-Aspartate + | Povidone (PVP K 25) | 16.5 |
| Carbonate Salt Granules) | Color FD&C YELLOW 6 | 6.5 |
| | ALUM Lake | |
| | Sodium Bicarbonate | 950.0 |
| | Weight of Granules | 1973.0 |
| Part-B | Aspartame | 90.0 |
| (Acidifier in dry mix) | Flavor: Orange 501071 | 50.0 |
| | Isomalt (galen IQ721) | 62.0 |
| | Citric Acid Anhydrous | 1525.0 |
| | Weight of dry mix | 1727.0 |

| | |
|---|---|
| TOTAL WEIGHT OF PART-A & B | 3700.0 |

### Detailed Method of Preparation of Samples

### Examples B_{C}&C_{C}

L-Ornithine L-Aspartate was granulated using purified water, dried and sized to mix with either Citric acid (example B) or Sodium bicarbonate (example C)

### Example D_{C}

L-Ornithine L-Aspartate was granulated along with Citric acid and Sodium bicarbonate using purified water, dried and sized.

### Examples E_{C}&F_{I}

L-Ornithine L-Aspartate was granulated using purified water either with Citric acid (example-E) or Sodium bicarbonate (example-F) dried and sized. Such granules were further mixed with Sodium bicarbonate (example-E) or Citric acid (example-F).

### Example G_{I}

L-Ornithine L-Aspartate, Povidone PVP K25, Color and Sodium bicarbonate were accurately weighed, mixed and granulated with water. Dried granules (Part-A) were sized and mixed with Aspartame, orange flavor, Isomalt and citric acid anhydrous (Part-B).

Resulting mixed granules were finally packed in HDPE bottles for stability studies carried out for the period of 12 days at 40° C./75% RH (relative humidity).

TABLE 4
Impurity results: Effect of method of incorporation of acidifier
and gas generating agent on stability of L-Ornithine L-Aspartate
granules (*-> Total impurities are expressed in parts per
million (PPM))

| | | Total Impurities (PPM)* | |
|---|---|---|---|
| | Qualitative Composition & | | 12 days, |
| Sample | process | Initial | 40° C./75% RH |
| A_{C} | Aqueous Granulation of | 50 | 50 |
| | L-Ornithine L-Aspartate | | |
| B_{C} | Aqueous Granulation of | 360 | 930 |
| | L-Ornithine L-Aspartate | | |
| | Extra Granular addition of | | |
| | Citric acid Anhydrous | | |
| C_{C} | Aqueous Granulation of | 110 | 270 |
| | L-Ornithine L-Aspartate | | |
| | Extra Granular addition of | | |
| | NaHCO3 | | |
| D_{C} | Aqueous Granulation together of | 150 | 470 |
| | L-Ornithine L-Aspartate | | |
| | Sodium Bicarbonate and | | |
| | Citric acid Anhydrous | | |
| E_{C} | Aqueous Granulation of | 80 | 1060 |
| | L-Ornithine L-Aspartate and | | |
| | Citric acid Anhydrous | | |
| | Extra Granular addition of | | |
| | Sodium Bicarbonate | | |
| F_{I} | Aqueous Granulation of | 90 | 150 |
| | L-Ornithine L-Aspartate and | | |
| | Sodium Bicarbonate | | |
| | Extra Granular addition of | | |
| | Citric acid | | |
| G_{I} | Aqueous Granulation of | 60 | 170 |
| | Part-A ingredients | | |
| | Extra Granular addition of | | |
| | Part-B ingredients | | |

Remarks on impurities in examples A_{C} to G_{I}: Impurities generated in inventive examples (F_{I} to G_{I}) was lower than that of comparative examples (B_{C} to E_{C}).

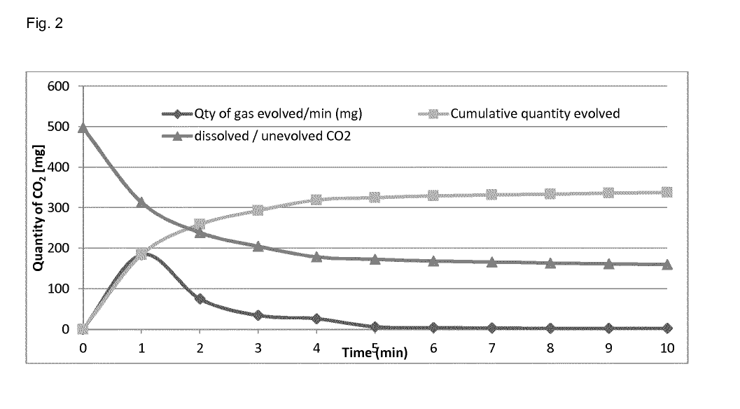

Sample-G_{I} was analyzed for effervescence/CO₂ generation characteristics in water (100 mL) at 25° C. (cf. FIG. 2).

TABLE 5
Effervescence/CO₂ generation characteristics of sample G_{I}

| Calculated CO₂ | Time for | Amount of | Amount of CO₂ in |
|---|---|---|---|
| generation | complete | CO₂ evolved | solution after |
| capacity (i) | effervescence | in 10 minutes (ii) | 10 min (i-ii) |
| 497 mg | 4 min | 338 mg | 160 mg |

Considering that the solubility of Carbon dioxide in water at 25° C. is about 1.45 g/L (as reported in Wikipedia), sample-G could supersaturate the drink with carbon dioxide giving enough carbonation for imparting good taste.

Based on above data additional experimentation was planned to further study effect of process and amount of acidifier in the formulations by adopting above processes

TABLE 6
Examples H_{I}, I_{I} and J_{I} (inventive)

| | | Weight per dose (mg) | | |
|---|---|---|---|---|
| | | Example- | Example- | Example- |
| | Ingredients | H_{I} | I_{I} | J_{I} |
| Part-A | L-Ornithine L-Aspartate | 1000.0 | 1000.0 | 1000.0 |
| (L-Ornithine L- | Povidone (PVP K 25) | 16.5 | 16.5 | 16.5 |
| Aspartate + | Color FD&C YELLOW 6 ALUM | 6.5 | 6.5 | 6.5 |
| Carbonate salt | Lake | | | |
| Granules) | Sodium Bicarbonate | 950.0 | 950.0 | 950.0 |
| | Weight of Granules | 1973.0 | 1973.0 | 1973.0 |
| Part-B | Aspartame | 90.0 | 90.0 | 90.0 |
| (Acidifier in dry mix) | Flavor: Orange 501071 | 50.0 | 50.0 | 50.0 |
| | Isomalt (galen IQ721) | 62.0 | 62.0 | 62.0 |
| | Citric Acid Anhydrous | 1525.0 | 724.4 | 344.0 |
| | Weight of dry mix | 1727.0 | 926.4 | 546.0 |

| | | | |
|---|---|---|---|
| TOTAL WEIGHT OF PART-A & B | 3700.0 | 2899.4 | 2519.0 |

### Detailed Method of Preparation for Examples—H_{I}, L_{I} and J_{I}

Part-A: L-Ornithine L-Aspartate, Povidone PVP K25, Color and Sodium bicarbonate were accurately weighed, mixed and granulated with water in a rapid mixer granulator. Granules were dried in a fluidized bed processor to the LOD of <1%. Dried granules were sized to get #25 ASTM passing granules.

Part-B: Aspartame, flavor, Isomalt and Citric acid anhydrous were passed thru #25 ASTM sieve.

Part-A and B were mixed together for 5 minutes.

TABLE 7
Formula for examples K_{C}, L_{C} and M_{C} (Comparative)

| | | Weight per dose (mg) | | |
|---|---|---|---|---|
| | | Example- | Example- | Example- |
| | Ingredients | K_{C} | L_{C} | M_{C} |
| Part-A | L-Ornithine L-Aspartate | 1000.0 | 1000.0 | 1000.0 |
| (L-Ornithine L- | Povidone (PVP K 25) | 16.5 | 16.5 | 16.5 |
| Aspartate + acidifier | Color FD&C YELLOW 6 ALUM | 6.5 | 6.5 | 6.5 |
| Granules) | Lake | | | |
| | Citric Acid Anhydrous | 1525.0 | 724.4 | 344.0 |
| | Weight of Granules | 2548.0 | 1747.4 | 1367.0 |
| Part-B | Aspartame | 90.0 | 90.0 | 90.0 |
| (Carbonate salt in | Flavor: Orange 501071 | 50.0 | 50.0 | 50.0 |
| dry mix) | Isomalt (galen IQ721) | 62.0 | 62.0 | 62.0 |
| | Sodium Bicarbonate | 950.0 | 950.0 | 950.0 |
| | Weight of dry mix | 1152.0 | 1152.0 | 1152.0 |

| | | | |
|---|---|---|---|
| TOTAL WEIGHT OF PART-A & B | 3700.0 | 2899.4 | 2519.0 |

### Detailed Method of Preparation for Examples—K_{C}, L_{C} and M_{C}

Part-A: L-Ornithine L-Aspartate, Povidone PVP K25, Color and Citric acid were accurately weighed, mixed and granulated with water in a rapid mixer granulator. Granules were dried in a fluidized bed processor to the LOD of <1%. Dried granules were sized to get #25 ASTM passing granules.

Part-B: Aspartame, flavor, Isomalt and Sodium bicarbonate were passed thru #25 ASTM sieve.

Part-A and B were mixed together for 5 minutes.

These samples (H to M) were tested for impurity generation on storage. The results are mentioned in table 8 below.

TABLE 8
Impurity data for samples H to M

Total Impurities (PPM)*

| Sample | Initial | 12 days, 40° C./75% RH |
|---|---|---|
| H_{I} | 90 | 130 |
| I_{I} | 70 | 110 |
| J_{I} | 100 | 110 |
| K_{C} | 70 | 1500 |
| L_{C} | 60 | 2560 |
| M_{C} | 70 | 3270 |

Remarks on impurities in examples H_{I} to M_{C}: Impurities generated in inventive examples (H_{I} to J_{I}) were significantly lower than those of comparative examples (K_{C} to M_{C}). This signifies the importance of method of addition of the acidic and gas generating components.

Based on above results, additional experiments (N_{I}, O_{C} and P_{I}) were planned to evaluate effervescence characteristics using different inherent gas generation capacities of the formulations. Additionally example Q c was planned to study comparative properties of the prior art (CN103860517)

TABLE 9
Examples N_{I}, O_{C} and P_{I}

| | | Weight per dose (mg) | | |
|---|---|---|---|---|
| | Ingredients | Example-N_{I} | Example-O_{C} | Example-P_{I} |
| Part-A | L-Ornithine L- | 1000.0 | 1000.0 | 700 |
| (L-Ornithine L- | Aspartate | | | |
| Aspartate + | Povidone (PVP K 25) | 16.5 | 16.5 | 11.55 |
| Carbonate salt | Color FD&C YELLOW | 6.5 | 6.5 | 4.55 |
| Granules) | Sodium Bicarbonate | 950.0 | 950.0 | 665.0 |
| | Weight of Granules | 1973.0 | 1973.0 | 1381.1 |
| Part-B | Aspartame | 90.0 | 90.0 | 90.0 |
| (Acidifier in dry | Flavor: Orange | 50.0 | 50.0 | 50.0 |
| mix) | 501071 | | | |
| | Isomalt (galen IQ721) | 62.0 | 62.0 | 62.0 |
| | Citric Acid Anhydrous | 1525.0 | 218.0 | 1070.0 |
| | Weight of dry mix | 1727.0 | 420.0 | 1272.0 |
| Lubricants for | Polyethylene Glycol | 50.0 | — | — |
| tabletting | 6000 | | | |
| | L-Leucine | 50.0 | — | — |

| | | | |
|---|---|---|---|
| TOTAL WEIGHT OF PART-A & B | 3800.0 | 2393.0 | 3653.1 |

### Detailed Method of Preparation for Examples—N_{I}, O_{C}, P_{I}

Part-A: L-Ornithine L-Aspartate, Povidone PVP K25, Color and Sodium bicarbonate were accurately weighed, mixed and granulated with water in a rapid mixer granulator. Granules were dried in a fluidized bed processor to the LOD of <1%. Dried granules were sized to get #25 ASTM passing granules.

Part-B: Aspartame, flavor, Isomalt and Citric acid anhydrous were passed thru #25 ASTM sieve.

Part-A and B were mixed together for 5 minutes.

In case of example-N; part-A, part-B, PEG 6000 and L-Leucine were mixed together and compressed into tablets using 25 mm tablet tooling.

TABLE 10
Example Q_{C} (As per CN103860517)

| | | Weight per dose |
|---|---|---|
| | | (mg) |
| | Ingredients | Example Q |
| Part-A | L-Ornithine L-Aspartate | 1000.0 |
| (L-Ornithine L- | Kollidon VA 64 | 45.0 |
| Aspartate + tartaric | Tartaric acid | 338.0 |
| acid granules) | Ethanol (q.s. to 8% Povidone | Q.S |
| | solution) | |
| | Weight of Granules | 1383.0 |
| Part-B | Sodium Bicarbonate | 210.0 |
| (Carbonate + PEG | PEG 6000 | 130.0 |
| granulate) | Weight of melt granulates | 340.0 |
| Dry excipients | Aspartame | 0.75 |
| | Flavor | 0.75 |

| | |
|---|---|
| TOTAL WEIGHT OF PART-A, B &dry excipients | 1724.5 |

### Detailed Method of Preparation for Example—Q_{C}

Part-A: L-Ornithine L-Aspartate and tartaric acid mixed together. Kollidon VA 64 dissolved in Ethanol to give 8% solution. This solution was used to granulate powder mixture and dried. Dried granules were sized to get #25 ASTM passing granules.

Part-B: PEG 6000 was mixed with sodium bicarbonate and melt granulated at 60 deg C. in water bath. Cooled and illed to get were passed thru #25 ASTM sieve.

Part-A and B were mixed together for 5 minutes.

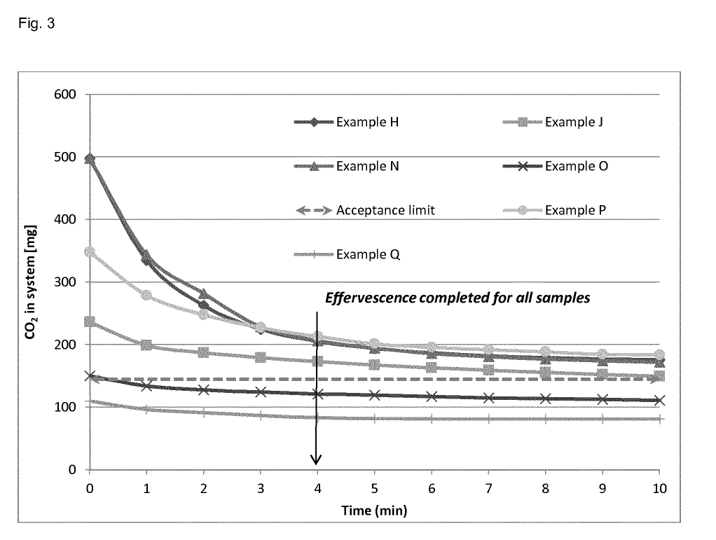

Samples H_{I}, J_{I}, N_{I} O_{C}, P_{I}, Q_{C} were analyzed for effervescence/CO₂ generation characteristics in water (100 mL) at 25° C. (cf. FIG. 3).

TABLE 11
Effervescence/CO₂ generation characteristics
of samples in water (100 mL) at 25° C.

| | | | Amount of | Amount of CO₂ |
|---|---|---|---|---|
| | Calculated CO₂ | Time for | CO₂ | in solution after |
| | generation | complete | evolved in | 10 min |
| Sample | capacity (i) | effervescence | 10 min (ii) | (i-ii) |
| Q_{C} | 110 mg | 2 min | 29 mg | 81 mg |
| O_{C} | 150 mg | 2 min | 39 mg | 111 mg |
| J_{I} | 236 mg | 4 min | 86 mg | 150 mg |
| P_{I} | 348 mg | 4 min | 164 mg | 184 mg |
| H_{I} | 497 mg | 4 min | 122 mg | 176 mg |
| N_{I} | 497 mg | 4 min | 326 mg | 172 mg |

Remarks on effervescence characteristics in examples H_{I}, J_{I} and N_{I} to Q_{C}: Considering that the solubility of Carbon dioxide in water at 25° C. is about 1.45 g/L (as reported in Wikipedia), all samples excluding those of the comparative examples (O_{C} and Q_{C}) could supersaturate the drink with carbon dioxide giving enough carbonation for imparting good taste.

The impurity generation results for examples H_{I}, N_{I} and Q_{C} are in table below.

TABLE 12
Impurity data for samples H_{I}, N_{I} and Q_{C}

Total Impurities (PPM)*

| Sample | Initial | 12 days, 40° C./75% RH |
|---|---|---|
| H_{I} | 90 | 130 |
| N_{I} | 50 | 60 |
| Q_{C} | 100 | 3090 |

Remarks on impurities in examples H_{I}, N_{I} and Q_{C}: Impurities generated in inventive examples (H_{I} and N_{I}) were significantly lower than those of comparative example from prior art (Q_{C}). This signifies the importance of method of addition of the acidic and gas generating components.

Additional examples were studied to ascertain the effect of change in type of acid on impurity generation behavior of the invention.

TABLE 13
Example R_{I} (replacement of Citric acid with
tartaric acid) Positive example

| | | Weight per dose (mg) |
|---|---|---|
| | Ingredients | Example-R_{I} |
| Part-A | L-Ornithine L-Aspartate | 1000.0 |
| (L-Ornithine L- | Povidone (PVP K 25) | 16.5 |
| Aspartate + | Color FD&C YELLOW | 6.5 |
| Carbonate salt | Sodium Bicarbonate | 950.0 |
| Granules) | Weight of Granules | 1973.0 |
| Part-B | Aspartame | 90.0 |
| (Acidifier in dry mix) | Flavor: Orange 501071 | 50.0 |
| | Isomalt (galen IQ721) | 62.0 |
| | Tartaric Acid | 1525.0 |
| | Weight of dry mix | 1727.0 |

| | |
|---|---|
| TOTAL WEIGHT OF PART-A & B | 3700.0 |

### Detailed Method of Preparation for Example—R_{I}

Part-A: L-Ornithine L-Aspartate, Povidone PVP K25, Color and Sodium bicarbonate were accurately weighed, mixed and granulated with water in a rapid mixer granulator. Granules were dried in a fluidized bed processor to the LOD of <1%. Dried granules were sized to get #25 ASTM passing granules.

Part-B: Aspartame, flavor, Isomalt and Tartaric acid were passed thru #25 ASTM sieve.

Part-A and B were mixed together for 5 minutes.

TABLE 14
Formula for example S_{C} (Comparative against example R_{I})

| | | Weight per |
|---|---|---|
| | | dose (mg) |
| | Ingredients | Example-S_{C} |
| Part-A | L-Ornithine L-Aspartate | 1000.0 |
| (L-Ornithine L- | Povidone (PVP K 25) | 16.5 |
| Aspartate + acidifier | Color FD&C YELLOW 6 ALUM | 6.5 |
| Granules) | Lake | |
| | Tartaric Acid | 1525.0 |
| | Weight of Granules | 2548.0 |
| Part-B | Aspartame | 90.0 |
| (Carbonate salt in | Flavor: Orange 501071 | 50.0 |
| dry mix) | Isomalt (galen IQ721) | 62.0 |
| | Sodium Bicarbonate | 950.0 |
| | Weight of dry mix | 1152.0 |

| | |
|---|---|
| TOTAL WEIGHT OF PART-A & B | 3700.0 |

### Detailed Method of Preparation for Example—S_{C}

Part-A: L-Ornithine L-Aspartate, Povidone PVP K25, Color and Tartaric acid were accurately weighed, mixed and granulated with water in a rapid mixer granulator. Granules were dried in a fluidized bed processor to the LOD of <1%. Dried granules were sized to get #25 ASTM passing granules.

Part-B: Aspartame, flavor, Isomalt and Sodium bicarbonate were passed thru #25 ASTM sieve.

Part-A and B were mixed together for 5 minutes.

TABLE 15
Impurity data for samples R_{I} and S_{C}

| | |
|---|---|
| Results of impurity | |
| analysis of examples R_{I} | |
| and S_{C} | Total Impurities (PPM)* |

| Sample | Initial | 12 days, 40° C./75% RH |
|---|---|---|
| R_{I} | 150 | 780 |
| S_{C} | 210 | 14470 |

Remarks on impurities in examples R_{I} and S_{C}: Impurities generated in inventive example (R_{I}) was significantly lower than that of comparative examples (S_{C}). Thus as expected, in spite of change in type of acid, the impurity generation behavior of the formulations remain unaffected.

In order to study applicability of the invention to the type of granulation techniques, additional experiments (T_{I} and U_{I}) were planned. Effect of melt granulation and dry granulation was studied.

TABLE 16
Examples T_{I} and U_{I} (inventive, demonstrating
dry and melt granulation techniques)

| | | Weight per dose (mg) | |
|---|---|---|---|
| | | Example-T_{I} | Example-U_{I} |
| | | Dry | Melt |
| | Ingredients | Granulation | granulation |
| Part-A | L-Ornithine L-Aspartate | 1000.0 | 1000.0 |
| (L-Ornithine L- | Povidone (PVP K 25) | 16.5 | — |
| Aspartate + | PEG 6000 | — | 200.0 |
| Carbonate salt | Color FD&C YELLOW | 6.5 | 6.5 |
| Granules) | Sodium Bicarbonate | 950.0 | 950.0 |
| | Weight of Granules | 1973.0 | 2156.5 |
| Part-B | Aspartame | 90.0 | 90.0 |
| (Acidifier in | Flavor: Orange 501071 | 50.0 | 50.0 |
| dry mix) | Isomalt (galen IQ721) | 62.0 | 62.0 |
| | Citric Acid Anhydrous | 1525.0 | 1525.0 |
| | Weight of dry mix | 1727.0 | 1727.0 |

| | | |
|---|---|---|
| TOTAL WEIGHT OF PART-A & B | 3700.0 | 3883.5 |

### Detailed Method of Preparation

### Dry Granulation (Example T_{I})

Part-A: L-Ornithine L-Aspartate, Povidone PVP K25, Color and Sodium bicarbonate were accurately weighed, passed through #40 ASTM sieve and mixed for 5 minutes. Resulting blend was compacted on to obtain compacts. The compacts were milled and sized through #25 ASTM sieve.

Part-B: Aspartame, flavor, Isomalt and Citric acid anhydrous were passed thru #25 ASTM sieve.

Part-A and B were mixed together for 5 minutes.

### Melt Granulation (Example U_{I})

Part-A: L-Ornithine L-Aspartate, PEG 6000, colour and Sodium bicarbonate were accurately weighed and passed through #40 ASTM sieve and mixed. Resulting blend mix was transferred to glass beaker and heated at 65° C. on hot plate and mixed to ensure homogeneous mix. After complete melting of PEG 6000 melted mass was removed, cooled and sifted through #25 ASTM sieve.

Part-B: Aspartame, flavor, Isomalt and Citric acid anhydrous were passed thru #25 ASTM sieve.

Part-A and B were mixed together for 5 minutes.

Samples, T_{I} and U_{I} were tested for impurity generation on storage. The results are in table below.

TABLE 17
Impurity data for samples T_{I} and U_{I}

Total Impurities (PPM)*

| Sample | Initial | 12 days, 40° C./75% RH |
|---|---|---|
| T_{I} | 70 | 290 |
| U_{I} | 40 | 100 |

Remarks on impurities in examples T_{I} and U_{I}: Impurities generated in both the inventive examples (T_{I} and U_{I}) was not increased significantly and the behavior was similar to other inventive examples mentioned earlier. Thus, in spite of change in type of granulation technique, the impurity generation behavior of the formulations remain unaffected.

Another experiment (sample V_{I}) explained below was planned by using ascorbic acid as acidic component and Elderberry extract as color and flavor.

TABLE 18
Formula for example V_{I}

| | | Weight per dose (mg) |
|---|---|---|
| | Ingredients | Example-V_{I} |
| Part-A | L-Ornithine L-Aspartate | 1000.0 |
| (L-ornithine L- | Sodium Bicarbonate | 700.0 |
| aspartate + | Weight of Granules | 1700.0 |
| Carbonate salt | | |
| Granules) | | |
| Part-B | Aspartame | 180.0 |
| (Acidifier in dry mix) | Elderberry extract | 400 |
| | Isomalt (galen IQ721) | 1720 |
| | Ascorbic acid | 2000 |
| | Weight of dry mix | 6000.0 |

### Detailed Method of Preparation for Example—V

Part-A: L-Ornithine L-Aspartate and Sodium bicarbonate were accurately weighed, mixed and granulated with water in a rapid mixer granulator. Granules were dried in a fluidized bed processor to the LOD of <1%. Dried granules were sized to get #25 ASTM passing granules.

Part-B: Aspartame, Elderberry extract, Isomalt and Ascorbic acid were passed thru #30 ASTM sieve. Part-A and B were mixed together for 5 minutes.

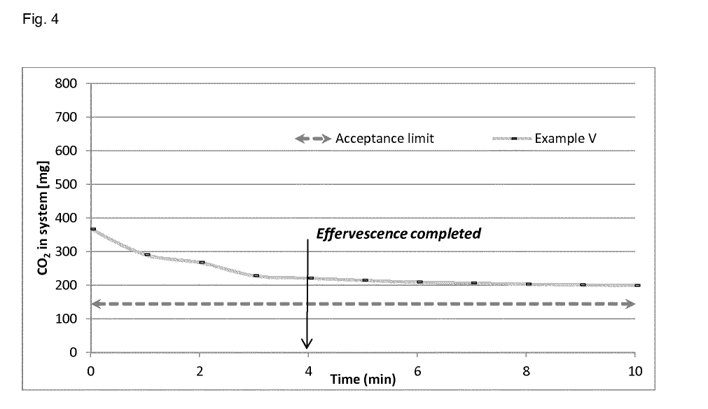

Sample-V_{I} was analyzed for effervescence/CO₂ generation characteristics in water (100 mL) at 25° C. (cf. FIG. 4).

TABLE 19
Effervescence/CO₂ generation characteristics
of sample V in water (100 mL) at 25° C.

| | | | | Amount |
|---|---|---|---|---|
| | Calculated | | | of CO₂ |
| | CO₂ | | | in |
| | generation | Time for | Amount of CO₂ | solution |
| | capacity | complete | evolved in 10 min | after 10 min |
| Sample | (i) | effervescence | (ii) | (i-ii) |
| V | 367 mg | 4 min | 168 mg | 199 mg |

Remarks on effervescence characteristics in example V_{I}: As observed with other inventive examples, example V_{I} exhibited acceptable effervescence generating characteristics. No significant effect of change in acidic component, color or flavor was observed.

1: A process for manufacturing an effervescent formulation of ornithine aspartate, the process comprising:
granulating an ornithine aspartate-mix that comprises ornithine aspartate and a gas generating component comprising one or more gas generating agents, thus yielding granules G, and
mixing components of a final mix that comprises the granules G and an acid component comprising a pharmaceutically acceptable acid, thus yielding the effervescent formulation of ornithine aspartate.

2: The process according to claim 1, wherein the granulating is performed under a granulation condition selected from the group consisting of wet granulation, dry granulation, and melt granulation.

3: The process according to claim 1, wherein a total amount of the gas generating agents in the gas generating component added to the formulation is sufficient to yield effervescent formulations comprising 20 wt % to 40 wt % of gas generating component.

4: The process according to claim 1, wherein the one or more gas generating agents in the gas generating component are selected from the group consisting of a carbonate salt, a bicarbonate salt, and a mixture thereof.

5: The process according to claim 1, wherein the cane or more gas generating agents in the gas generating component are selected from the group consisting of sodium carbonate, sodium bicarbonate, potassium carbonate, potassium bicarbonate, magnesium carbonate, and calcium carbonate.

6: The process according to claim 1, wherein the gas generating component consists of sodium bicarbonate.

7: The process according to claim 1, wherein the pharmaceutically acceptable acid in the acid component is selected from the group consisting of citric acid, tartar acid, malic acid, adipic acid, succinic acid, fumaric acid, ascorbic acid, maleic acid, a partial salt with an alkaline or an alkaline earth metal of an above-mentioned polybasic acid, and a mixture thereof.

8: The process according to claim 1, wherein the acid component consists of citric acid.

9: An effervescent formulation of ornithine aspartate, obtained by the process according to claim 1.

10: The effervescent formulation of ornithine aspartate according to claim 9, comprising one or more excipients selected from the group consisting of a viscosity modifier, a filler, a disintegrant, a lubricant, a diluent, a binder, a glidant, an antifoaming agent, a wetting agent, a colorant, a sweetener, and a flavorant.

11: The effervescent formulation of ornithine aspartate according to claim 9, exhibiting a gas generating capacity of more than 150 mg carbon dioxide per gram of ornithine aspartate.

12: A tablet, pellet, or sachet, comprising the effervescent formulation of ornithine aspartate according to claim 9.
